(19) United States
(12) Patent Application Publication
(10) Pub. No.: US 2010/0159521 A1
(43) Pub. Date: Jun. 24, 2010

(54) OZONE TREATMENT OF BIOMASS TO ENHANCE ENZYMATIC SACCHARIFICATION

(75) Inventors: Jelena Cirakovic, Wilmington, DE (US); Bruce A. Diner, Chadds Ford, PA (US)

Correspondence Address: E I DU PONT DE NEMOURS AND COMPANY;LEGAL PATENT RECORDS CENTER, BARLEY MILL PLAZA 25/1122B, 4417 LANCASTER PIKE, WILMINGTON, DE, 19805, US

(73) Assignee: E. I. DU PONT DE NEMOURS AND COMPANY, Wilmington, DE (US)

(21) Appl. No.: 12/640,088

(22) Filed: Dec. 17, 2009

Related U.S. Application Data
(60) Provisional application No. 61/139,116, filed on Dec. 19, 2008.

(51) Int. Cl. C12P 19/00; C07H 1/08

(52) U.S. Cl. 435/72; 536/128

(57) ABSTRACT

Methods for treating lignocellulosic biomass to produce readily saccharifiable carbohydrate-enriched biomass are provided. In one method, lignocellulosic biomass comprising lignin is treated with aqueous ammonia, then contacted with a gas comprising ozone at a temperature of about 0° C. to about 50° C. In another method, lignocellulosic biomass comprising lignin is contacted with a gas comprising ozone at a temperature of about 0° C. to about 50° C., then treated with aqueous ammonia. The readily saccharifiable carbohydrate-enriched biomass may be saccharified with an enzyme consortium to produce fermentable sugars.

### CROSS-REFERENCE TO RELATED APPLICATION

This application claims benefit of priority from Provisional Application No. 61/139,116 filed Dec. 19, 2008. This application hereby incorporates by reference Provisional Application No. 61/139,116 in its entirety.

### FIELD OF THE INVENTION

Methods for producing readily saccharifiable, carbohydrate-enriched lignocellulosic biomass are provided and disclosed. Specifically, pretreated biomass may be prepared by treating under conditions of high solids and low ammonia concentration, then by contacting with a gas comprising ozone. The remaining carbohydrate-enriched solids in the pretreated biomass may then be subjected to enzymatic saccharification to obtain fermentable sugars, which may be subjected to further processing for the production of other target products.

### BACKGROUND OF THE INVENTION

Cellulosic and lignocellulosic feedstocks and wastes, such as agricultural residues, wood, forestry wastes, sludge from paper manufacture, and municipal and industrial solid wastes, provide a potentially large renewable feedstock for the production of chemicals, plastics, fuels and feeds. Cellulosic and lignocellulosic feedstocks and wastes, composed of carbohydrate polymers comprising cellulose, hemicellulose, pectins and lignin are generally treated by a variety of chemical, mechanical and enzymatic means to release primarily hexose and pentose sugars, which can then be fermented to useful products.

Pretreatment methods are usually used to make the polysaccharides of lignocellulosic biomass more readily accessible to cellulolytic enzymes. One of the major impediments to cellulolytic enzyme digest of polysaccharide is the presence of lignin, a barrier that limits the access of the enzymes to their substrates, and a surface to which the enzymes bind non-productively. Because of the significant cost of enzyme in the pretreatment process, it is desirable to minimize the enzyme loading by either inactivation of the lignin to enzyme adsorption or its outright extraction. Another challenge is the inaccessibility of the cellulose to enzymatic hydrolysis either because of its protection by hemicellulose and lignin or by its crystallinity. Pretreatment methods that attempt to overcome these challenges include: steam explosion, hot water, dilute acid, ammonia fiber explosion, alkaline hydrolysis (including ammonia recycled percolation), oxidative delignification, organosolv, and ozonation.

Previously applied pretreatments methods often suffer from shortcomings, including separate hexose and pentose streams (e.g. dilute acid), inadequate lignin extraction or lack of separation of extracted lignin from polysaccharide, particularly in those feedstocks with high lignin content (e.g., sugar cane bagasse, softwoods), disposal of waste products (e.g., salts formed upon neutralization of acid or base), and poor recoveries of carbohydrate due to breakdown or loss in wash steps. Other problems include the high cost of energy, capital equipment, and pretreatment catalyst recovery, and incompatibility with saccharification enzymes.

Ben-Ghedalia et al. (in J. Sci. Food Agric. 1980, 31(12), 1337-1342) disclose treatment of cotton straw with ammonium hydroxide (at room temperature for 60 days), ozone treatment, and combined ammonium hydroxide treatment followed by ozonation. They report that ozone treatment caused a 50% reduction in lignin and hemicellulose, and a corresponding increase in cell contents. In vitro organic matter digestibility, as measured by the rumen liquor-acid pepsin method, was increased by more than 100% as a result of the partial conversion of cell walls into cell contents and the increased digestibility of the cell walls. Cellulose in vitro digestibility was increased by the combined treatment as well. No information on sugar recovery was provided.

One of the major challenges of the pretreatment of lignocellulosic biomass is to maximize the extraction or chemical neutralization (with respect to non-productive binding of cellulolytic enzymes) of the lignin while minimizing the loss of carbohydrate (cellulose plus hemicellulose). The higher the selectivity, the higher the overall yield of monomeric sugars following combined pretreatment and enzymatic saccharification.

### SUMMARY OF THE INVENTION

The present invention provides methods for producing readily saccharifiable carbohydrate-enriched biomass and for selectively oxidizing lignin while retaining carbohydrate in good yield. The methods include treating lignocellulosic biomass under conditions of high solids and low ammonia concentration, then contacting the biomass with a gas comprising ozone. The methods also include contacting lignocellulosic biomass with a gas comprising ozone, then treating the biomass under conditions of high solids and low ammonia concentration. With these methods, carbohydrate-enriched biomass, highly susceptible to enzymatic saccharification, is produced in a cost effective process. Following pretreatment, the carbohydrate-enriched biomass may be further treated with a saccharification enzyme consortium to produce high yields of fermentable sugars (for example, glucose and xylose). These sugars may be subjected to further processing, such as bioconversion to value-added chemicals and fuels.

In one embodiment of the invention, a method is provided, the method comprising:

(a) providing lignocellulosic biomass comprising lignin;

(b) contacting the biomass with an aqueous solution comprising ammonia to form a biomass-aqueous ammonia mixture, wherein the ammonia is present at a concentration at least sufficient to maintain alkaline pH of the biomass-aqueous ammonia mixture but wherein said ammonia is present at less than about 12 weight percent relative to dry weight of biomass, and further wherein the dry weight of biomass is at a high solids concentration of at least about 15 weight percent relative to the weight of the biomass-aqueous ammonia mixture, to produce an ammonia-treated biomass; and

(c) contacting the ammonia-treated biomass with a gas comprising ozone at a temperature of about 0° C. to about 50° C. whereby a readily saccharifiable carbohydrate-enriched biomass is produced.

In one embodiment of the invention a method is provided, the method comprising:

(a) providing lignocellulosic biomass comprising lignin;

(b) contacting the biomass with a gas comprising ozone at a temperature of about 0° C. to about 50° C.

(c) contacting the ozone-treated biomass with an aqueous solution comprising ammonia to form a mixture comprising ozone-treated biomass and aqueous ammonia, wherein the ammonia is present at a concentration at least sufficient to maintain alkaline pH of the mixture but wherein said ammonia is present at less than about 12 weight percent relative to dry weight of ozone treated biomass, and further wherein the dry weight of biomass is at a high solids concentration of at least about 15 weight percent relative to the weight of the mixture, whereby a readily saccharifiable carbohydrate-enriched biomass is produced.

According to the methods of the invention, the gas comprises about 0.1 to about 20 percent by volume ozone. In some embodiments, the gas comprises about 0.5 to about 5 percent by volume ozone. In some embodiments, the gas further comprises air, nitrogen, oxygen, argon, or a combination thereof. In some embodiments, the ratio of ozone to ammonia-treated biomass is at least 1:500 on a weight basis. In some embodiments, the ratio of ozone to lignocellulosic biomass is at least 1:100 on a weight basis. According to the methods of the invention, in some embodiments the temperature is about 0° C. to about 25° C.

In some embodiments, the methods further comprise applying energy to the lignocellulosic biomass, to the ammonia-treated biomass, or to both. In some embodiments, the methods further comprise applying energy to the lignocellulosic biomass, to the ozone-treated biomass, or to both. The applying energy is by milling, crushing, grinding, shredding, chopping, disc refining, ultrasound, microwave, or a combination of these. In some embodiments, the lignocellulosic biomass, the ammonia-treated biomass, or both contains at least about 30 percent moisture.

In some embodiments, the methods of the invention further comprise saccharifying the biomass with an enzyme consortium whereby fermentable sugars are produced. In some embodiments, the methods of the invention further comprise fermenting the sugars to produce a target product. In some embodiments, the target product is selected from the group consisting of ethanol, butanol, and 1,3-propanediol.

In some embodiments, the pH of the biomass-aqueous ammonia mixture is greater than about 8. In some embodiments, the ammonia is present at less than about 10 weight percent relative to dry weight of biomass. In some embodiments, ammonia is selected from the group consisting of ammonia gas, ammonium hydroxide, urea, and combinations thereof. In some embodiments, the aqueous solution comprising ammonia further comprises at least one additional base selected from the group consisting of sodium hydroxide, sodium carbonate, potassium hydroxide, potassium carbonate, calcium hydroxide, and calcium carbonate.

### DETAILED DESCRIPTION OF THE INVENTION

The present invention provides methods for the treatment of biomass in order to enhance the subsequent enzymatic saccharification step. In one method, in the first step biomass at relatively high concentration is treated with a relatively low concentration of ammonia relative to the dry weight of the biomass. Then in the second step, the ammonia-treated biomass, as an aqueous suspension or as a solid, is contacted with a gas comprising ozone. The treated biomass may be digested with a saccharification enzyme consortium to produce fermentable sugars. In another method, in the first step biomass, as an aqueous suspension or as a solid, is contacted with a gas comprising ozone. Then in the second step, the ozone-treated biomass at relatively high concentration is treated with a relatively low concentration of ammonia relative to the weight of the biomass.

Applicants specifically incorporate the entire contents of all cited references in this disclosure. Further, when an amount, concentration, or other value or parameter is given as either a range, preferred range, or a list of upper preferable values and lower preferable values, this is to be understood as specifically disclosing all ranges formed from any pair of any upper range limit or preferred value and any lower range limit or preferred value, regardless of whether ranges are separately disclosed. Where a range of numerical values is recited herein, unless otherwise stated, the range is intended to include the endpoints thereof, and all integers and fractions within the range. It is not intended that the scope of the invention be limited to the specific values recited when defining a range.

### DEFINITIONS

The following definitions are used in this disclosure:

“Room temperature” and “ambient” when used in reference to temperature refer to any temperature from about 15° C. to about 25° C.

“Fermentable sugars” refers to a sugar content primarily comprising monosaccharides and some polysaccharides that can be used as a carbon source by a microorganism in a fermentation process to produce a target product.

“Lignocellulosic” refers to material comprising both lignin and cellulose. Lignocellulosic material may also comprise hemicellulose. In the methods described herein, lignin is oxidized and substantially degraded to produce a carbohydrate-enriched biomass comprising fermentable sugars.

“Dissolved lignin” means the lignin that is dissolved in a solvent.

“Al lignin” refers to acid-insoluble lignin.

“Autohydrolysis” refers to the hydrolysis of biomass in the presence of solvent (water or organic solvent plus water) plus heat with no further additions, such as without hydrolytic enzymes

“Cellulosic” refers to a composition comprising cellulose.

“Target product” refers to a chemical, fuel, or chemical building block produced by fermentation. Product is used in a broad sense and includes molecules such as proteins, including, for example, peptides, enzymes, and antibodies. Also contemplated within the definition of target product are ethanol and butanol.

The abbreviation “EtOH” refers to ethanol or ethyl alcohol.

“Dry weight of biomass” refers to the weight of the biomass having all or essentially all water removed. Dry weight is typically measured according to American Society for Testing and Materials (ASTM) Standard E1756-01 (Standard Test Method for Determination of Total Solids in Biomass) or Technical Association of the Pulp and Paper Industry, Inc. (TAPPI) Standard T-412 om-02 (Moisture in Pulp, Paper and Paperboard).

“Selective extraction” means removal of lignin while substantially retaining carbohydrates.

A “solvent” as used herein is a liquid that dissolves a solid, liquid, or gaseous solute, resulting in a solution.

“Biomass” and “lignocellulosic biomass” as used herein refer to any lignocellulosic material, including cellulosic and hemi-cellulosic material, for example, bioenergy crops, agricultural residues, municipal solid waste, industrial solid waste, yard waste, wood, forestry waste, and combinations thereof, and as further described below. Biomass has a carbohydrate content that comprises polysaccharides and oligosaccharides and may also comprise additional components, such as protein and/or lipid.

“Highly conserved” as used herein refers to the carbohydrate content of the lignocellulosic material after the processing steps described herein. In an embodiment of the invention, the highly conserved carbohydrate content provides for sugar yields after saccharification that are substantially similar to theoretical yields and/or demonstration of minimal loss in sugar yield from the processes described herein. In an embodiment of the invention, highly-conserved with reference to carbohydrate content refers to the conservation of greater than or equal to 85% of the biomass carbohydrate as compared to biomass prior to pretreating as described herein.

“Preprocessing” as used herein refers to processing of lignocellulosic biomass prior to pretreatment. Preprocessing is any treatment of biomass that prepares the biomass for pretreatment, such as mechanically chopping and/or drying to the appropriate moisture content.

“Aqueous ammonia-treated biomass suspension” refers to a mixture of ammonia-treated biomass and aqueous solution wherein the biomass is in suspension in the aqueous solution. The biomass suspension may comprise additional components such as a buffer. As used herein, “slurry” is used interchangeably with “suspension.”

“Saccharification” refers to the production of fermentable sugars from polysaccharides by the action of hydrolytic enzymes. Production of fermentable sugars from pretreated biomass occurs by enzymatic saccharification by the action of cellulolytic and hemicellulolytic enzymes.

“Pretreating biomass” or “biomass pretreatment” as used herein refers to subjecting native or preprocessed biomass to chemical, physical, or biological action, or any combination thereof, rendering the biomass more susceptible to enzymatic saccharification or other means of hydrolysis prior to saccharification. For example, the methods claimed herein may be referred to as pretreatment processes that contribute to rendering biomass more accessible to hydrolytic enzymes for saccharification.

“Pretreated biomass” as used herein refers to native or preprocessed biomass that has been subjected to chemical, physical, or biological action, or any combination thereof, rendering the biomass more susceptible to enzymatic saccharification or other means of hydrolysis prior to saccharification.

“Air-drying the filtered biomass” can be performed by allowing the biomass to dry through equilibration with the air of the ambient atmosphere.

“Readily saccharifiable biomass” means biomass that is carbohydrate-enriched and made more amenable to hydrolysis by cellulolytic or hemi-cellulolytic enzymes for producing monomeric and oligomeric sugars.

“Carbohydrate-enriched” as used herein refers to the biomass produced by the process treatments described herein in which lignin in the biomass is selectively oxidized and degraded while biomass carbohydrate is retained in good yield. In one embodiment the readily saccharifiable carbohydrate-enriched biomass produced by the processes described herein has a carbohydrate concentration of greater than or equal to about 85% of the biomass carbohydrate as compared to biomass prior to pretreating as described herein while removing 75% or greater of the biomass lignin.

“Filtering free liquid under pressure” means removal of unbound liquid through filtration, with some pressure difference on opposite faces of the filter.

“Air-dried sample” means a pretreated biomass which is allowed to dry at ambient temperature to the point where its moisture content is approximately in equilibrium with that of the ambient air, typically ≧85% dry matter.

“Substantially lignin-free biomass” means a pretreated sample containing about ≦25% of the starting lignin composition.

“Pressure vessel” is a sealed vessel that may be equipped or not with a mechanism for agitation of a biomass/solvent suspension, in which a positive pressure is developed upon heating the lignocellulosic biomass.

“Hydrolysate” refers to the liquid in contact with the lignocellulosic biomass which contains the products of hydrolytic reactions acting upon the biomass (either enzymatic or not), in this case monomeric and oligomeric sugars.

“Organosolv” means a mixture of organic solvent and water.

“Enzyme consortium” or “saccharification enzyme consortium” is a collection of enzymes, usually secreted by a microorganism, which in the present case will typically contain one or more cellulases, xylanases, glycosidases, ligninases and feruloyl esterases.

“Monomeric sugars” or “simple sugars” consist of a single pentose or hexose unit, e.g., glucose.

“Delignification” is the act of removing lignin from lignocellulosic biomass. In the context of this application delignification means fragmentation and degradation of lignin from the lignocellulosic biomass using ozone.

“Fragmentation” is a process in which lignocellulosic biomass is treated with ozone to break the lignin down into smaller subunits. In the context of the present application, oxidation of the lignin may contribute to breaking the lignin down into smaller subunits.

An “aqueous solution comprising ammonia” refers to the use of ammonia gas (NH₃), compounds comprising ammonium ions (NH₄⁺) such as ammonium hydroxide or ammonium sulfate, compounds that release ammonia upon degradation such as urea, and combinations thereof in an aqueous medium.

“Ozonation” is the act of treating biomass with ozone. The biomass may be present in an aqueous suspension or as a solid without an additional liquid phase.

Methods for pretreating lignocellulosic biomass to produce readily saccharifiable biomass are provided. These methods provide economic processes for rendering components of the lignocellulosic biomass more accessible or more amenable to enzymatic saccharification. In this disclosure, one pretreatment method involves first treating biomass under conditions of high solids and low ammonia concentration, then contacting the biomass with a gas comprising ozone; alternatively, in another method biomass may first be contacted with a gas comprising ozone, then treated under conditions of high solids and low ammonia concentration. The presence of ozone assists lignin fragmentation and carbohydrate recovery, and a readily saccharifiable carbohydrate-enriched biomass is produced.

In addition, the methods described in the present disclosure minimize the loss of carbohydrate during the pretreatment process and maximize the yield of monomeric sugars in saccharification.

### Lignocellulosic Biomass:

The lignocellulosic biomass pretreated herein includes, but is not limited to, bioenergy crops, agricultural residues, municipal solid waste, industrial solid waste, sludge from paper manufacture, yard waste, wood and forestry waste. Examples of biomass include, but are not limited to, corn cobs, crop residues such as corn husks, corn stover, grasses, wheat, wheat straw, barley, barley straw, hay, rice straw, switchgrass, waste paper, sugar cane bagasse, sorghum, soy, components obtained from milling of grains, trees, branches, roots, leaves, wood chips, sawdust, shrubs and bushes, vegetables, fruits, flowers and animal manure.

In one embodiment, biomass that is useful for the invention includes biomass that has a relatively high carbohydrate content, is relatively dense, and/or is relatively easy to collect, transport, store and/or handle.

In one embodiment of the invention, biomass that is useful includes corn cobs, corn stover, sugar cane bagasse and switchgrass.

In another embodiment, the lignocellulosic biomass includes agricultural residues such as corn stover, wheat straw, barley straw, oat straw, rice straw, canola straw, and soybean stover; grasses such as switch grass, miscanthus, cord grass, and reed canary grass; fiber process residues such as corn fiber, beet pulp, pulp mill fines and rejects and sugar cane bagasse; sorghum; forestry wastes such as aspen wood, other hardwoods, softwood and sawdust; and post-consumer waste paper products; as well as other crops or sufficiently abundant lignocellulosic material.

The lignocellulosic biomass may be derived from a single source, or biomass may comprise a mixture derived from more than one source; for example, biomass could comprise a mixture of corn cobs and corn stover, or a mixture of stems or stalks and leaves.

The biomass may be used directly as obtained from the source, or may be subjected to some preprocessing, for example, energy may be applied to the biomass to reduce the size, increase the exposed surface area, and/or increase the accessibility of lignin and of cellulose, hemicellulose, and/or oligosaccharides present in the biomass to the aqueous ammonia pretreatment, the ozonation pretreatment, and to saccharification enzymes. Energy means useful for reducing the size, increasing the exposed surface area, and/or increasing the accessibility of the lignin, and the cellulose, hemicellulose, and/or oligosaccharides present in the biomass to the aqueous ammonia pretreatment, the ozonation pretreatment, and to saccharification enzymes include, but are not limited to, milling, crushing, grinding, shredding, chopping, disc refining, ultrasound, and microwave. This application of energy may occur before or during either or both of the pretreatment steps, before or during saccharification, or any combination thereof.

Drying biomass prior to pretreatment may occur as well by conventional means, such as by using rotary dryers, flash dryers, or superheated steam dryers.

### Ammonia Treatment:

The concentration of ammonia used in the present pretreatment methods is minimally a concentration that is sufficient to maintain the pH of the biomass-aqueous ammonia mixture alkaline and maximally less than about 12 weight percent relative to dry weight of biomass. This low concentration of ammonia is sufficient for pretreatment, and the low concentration may also be less than about 10 weight percent relative to dry weight of biomass. A very low concentration of 6 percent ammonia relative to dry eight of biomass, or less, also may be used for the first pretreatment step. By alkaline is meant a pH of greater than 7.0. Particularly suitable is a pH of the biomass-aqueous ammonia mixture that is greater than 8. In one embodiment, ammonia is present at less than about 10 weight percent relative to dry weight of biomass. Particularly suitable is ammonia at less than about 6 weight percent relative to dry weight of biomass. In some embodiments, ammonia is selected from the group consisting of ammonia gas, ammonium hydroxide, urea, and combinations thereof.

Ammonia as used in one step of the present methods provides advantages over other bases. Ammonia partitions into a liquid phase and a vapor phase. Gaseous ammonia can diffuse more easily through biomass than a liquid base, resulting in more efficacious pretreatment at lower concentrations. Ammonia also is known to compete with hydrolysis, via ammonolysis, of acetyl esters in biomass to form acetamide.

Acetamide is less toxic than acetate to certain fermentation organisms, such as Zymomonas mobilis. See, for example, published patent application US 2007/0031918. Thus conversion of acetyl esters to acetamide rather than to acetic acid reduces the need to remove acetic acid. The use of ammonia also reduces the requirement to supplement growth medium used during fermentation with a nitrogen source.

In addition, ammonia is a low-cost material and thus provides an economical process. Ammonia can also be recycled to the pretreatment reactor during pretreatment or following pretreatment, thus enabling a more economical process. For example, following pretreatment with ammonia, as the temperature is decreased to that suitable for ozonation or saccharification, ammonia gas may be released, optionally in the presence of a vacuum, and may be recycled. In a continuous process, ammonia may be continuously recycled.

The aqueous solution comprising ammonia may optionally comprise at least one additional base, such as sodium hydroxide, sodium carbonate, potassium hydroxide, potassium carbonate, calcium hydroxide and calcium carbonate. The at least one additional base may be added in an amount that is combined with ammonium to form an amount of total base that is less than about 20 weight percent relative to biomass dry weight. Preferably the total second base plus ammonia is in an amount that is less than about 15 weight percent. Additional base(s) may be utilized, for example, to neutralize acids in biomass, to provide metal ions for the saccharification enzymes, or to provide metal ions for the fermentation growth medium.

In the present methods, the biomass dry weight is at an initial concentration of at least about 15% up to about 80% of the weight of the biomass-aqueous ammonia mixture. More suitably, the dry weight of biomass is at a concentration of from about 15% to about 60% of the weight of the biomass-aqueous ammonia mixture. The percent of biomass in the biomass-aqueous ammonia mixture is kept high to minimize the total volume of pretreatment material, making the process more economical. Keeping the percent biomass high also reduces the need for concentration of sugars resulting from saccharification of the pretreated biomass, for use in fermentation.

Pretreatment of biomass with ammonia solution may be carried out in any suitable vessel. Typically the vessel is one that can withstand pressure, has a mechanism for heating, and has a mechanism for mixing the contents. Commercially available vessels include, for example, the Zipperclave® reactor (Autoclave Engineers, Erie, Pa.), the Jaygo reactor (Jaygo Manufacturing, Inc., Mahwah, N.J.), and a steam gun reactor ((described in General Methods Autoclave Engineers, Erie, Pa.). Much larger scale reactors with similar capabilities may be used. Alternatively, the biomass and ammonia solution may be combined in one vessel, then transferred to another reactor. Also biomass may be pretreated in one vessel, then further processed in another reactor such as a steam gun reactor (described in General Methods; Autoclave Engineers, Erie, Pa.).

Prior to contacting the biomass with an aqueous solution comprising ammonia, vacuum may be applied to the vessel containing the biomass. By evacuating air from the pores of the biomass, better penetration of the solvent into the biomass may be achieved. The time period for applying vacuum and the amount of negative pressure that is applied to the biomass will depend on the type of biomass and can be determined empirically so as to achieve optimal pretreatment of the biomass (as measured by the production of fermentable sugars following saccharification).

The contacting of the biomass with an aqueous solution comprising ammonia may be carried out at a temperature of from about 4° C. to about 200° C. Initial contacting of the biomass with ammonia at 4° C., allowing impregnation at this temperature, was found to increase the efficiency of saccharification over non-pretreated native biomass. In another embodiment, contacting of the biomass may be carried out at a temperature of from about 75° C. to about 150° C. In still another embodiment, contacting of the biomass may be carried out at a temperature of from greater than about 90° C. to about 150° C.

The contacting of the biomass with an aqueous solution comprising ammonia may be carried out for a period of time up to about 25 hours. Longer periods of pretreatment are possible, however a shorter period of time may be preferable for practical, economic reasons. Typically a period of ammonia contact treatment may be about 8 hours or less. Longer periods may provide the benefit of reducing the need for application of energy for breaking up the biomass, therefore, a period of time up to about 25 hours may be preferable.

In one embodiment, the ammonia treatment step may be performed at a relatively high temperature for a relatively short period of time, for example at from about 100° C. to about 150° C. for about 5 minutes to about 2 hours. In another embodiment, the ammonia treatment step may be performed at a lower temperature for a relatively long period of time, for example from about 75° C. to about 100° C. for about 2 hours to about 8 hours. In still another embodiment, the pretreatment process may be performed at room temperature for an even longer period of time of about 24 hours. Other temperature and time combinations intermediate to these may also be used.

For the treatment with aqueous ammonia solution, the temperature, time for pretreatment, ammonia concentration, concentration of one or more additional reagents, biomass concentration, biomass type and biomass particle size are related; thus these variables may be adjusted as necessary to obtain an optimal product to be contacted with a gas comprising ozone or with a saccharification enzyme consortium, depending on the pretreatment method used.

The treatment with aqueous ammonia solution may be performed in any suitable vessel, such as a batch reactor or a continuous reactor. One skilled in the art will recognize that at higher temperatures (above 100° C.), a pressure vessel is required. The suitable vessel may be equipped with a means, such as impellers, for agitating the biomass-aqueous ammonia mixture. Reactor design is discussed in Lin, K.-H., and Van Ness, H. C. (in Perry, R. H. and Chilton, C. H. (eds), Chemical Engineer's Handbook, 5^{th} Edition (1973) Chapter 4, McGraw-Hill, NY). The pretreatment reaction may be carried out as a batch process, or as a continuous process.

It is well known to those skilled in the art that a nitrogen source is required for growth of microorganisms during fermentation; thus the use of ammonia during pretreatment provides a nitrogen source and reduces or eliminates the need to supplement the growth medium used during fermentation with a nitrogen source. If the pH of the pretreatment product exceeds that at which saccharification enzymes are active, or exceeds the range suitable for microbial growth in fermentation, acids may be utilized to reduce pH. The amount of acid used to achieve the desired pH may result in the formation of salts at concentrations that are inhibitory to saccharification enzymes or to microbial growth. In order to reduce the amount of acid required to achieve the desired pH and to reduce the raw material cost of NH₃ in the present pretreatment process, ammonia gas may be evacuated from the pretreatment reactor and recycled. Typically, at least a portion of the ammonia is removed, which reduces the pH but leaves some nitrogen that provides this nutrient for use in subsequent fermentation.

Alternatively, performing ozonation after ammonia pretreatment has its advantages, too. Ammonia is an inhibitor of hydrolytic enzymes-ozonation of ammonia-pretreated biomass will result in removal of residual ammonia, as ozone reacts with ammonia to yield nitrogen or nitrate. The resulting biomass would thus require less acid for adjusting the pH for subsequent steps (saccharification and fermentation). Typically, after ammonia treatment the biomass contains about 40 percent to about 60 percent moisture. If the biomass is dry, water may be added to adjust the moisture content to between about 30 percent and about 60 percent.

In order to obtain sufficient quantities of sugars from biomass, the biomass may be pretreated with an aqueous ammonia solution one time or more than one time. Similarly, an ozonation step or a saccharification reaction may be performed one or more times. Both pretreatment and saccharification processes may be repeated if desired to obtain higher yields of sugars. To assess performance of the pretreatment and saccharification processes, separately or together, the theoretical yield of sugars derivable from the starting biomass can be determined and compared to measured yields.

### Ozone Treatment:

According to the present methods, lignocellulosic biomass or ammonia-pretreated biomass is contacted with a gas comprising ozone. Ozone treatment promotes oxidation and fragmentation of the lignin and is beneficial to pretreatment, resulting in an increased accessibility of the carbohydrate-enriched biomass to enzymatic saccharification. The use of ozone as a means of lignin removal is relatively selective, leaving the carbohydrates largely intact. In addition, ozone (O₃) easily decomposes to oxygen (O₂) and water, leaving no residue from its use and contributing minimal atmospheric pollution.

The ozone may be generated by any means known in the art, for example from oxygen or air. In the present methods, the gas comprising ozone comprises about 0.1 to about 20 percent by volume ozone, for example about 0.5 to about 10 percent by volume ozone. The gas may further comprise nitrogen, oxygen, argon, or a combination thereof. The gas comprising ozone may also comprise one or more other gases as long as the presence or concentration of the other gases is not deleterious to the ozone treatment. Generally, the ratio of ozone to the pretreated biomass may be at least about 1:1200 on a weight basis, for example for example at least about 1:1000, or at least about 1:750, or at least about 1:500, or at least about 1:200, or at least about 1:100, or at least about 1:50.

In one method, lignocellulosic biomass is contacted with an aqueous solution comprising ammonia, then the ammonia-treated biomass is contacted with a gas comprising ozone. In some embodiments, the ratio of ozone to the ammonia-treated biomass may be at least about 1:500 on a weight basis, for example at least about 2 mg of ozone per gram of biomass. In some embodiments the ratio of ozone to the ammonia-treated biomass may be at least about 1:100. In some embodiments, the amount of ozone in relation to the pretreated biomass may be from about 0.2 mg O₃/g biomass to about 10 mg O₃/g biomass. Other ratios may also be used. Preferred is a ratio of ozone to biomass which is sufficient to fragment lignin while retaining carbohydrate in good yield. Use of excess ozone beyond that which is optimal for delignification may lead to carbohydrate loss and lower sugar yields through saccharification.

In one method, lignocellulosic biomass is contacted with a gas comprising ozone, then the ozone-treated biomass is contacted with an aqueous solution comprising ammonia. In some embodiments, the ratio of ozone to the native lignocellulosic biomass may be at least about 1:100 on a weight basis, for example at least about 10 mg of ozone per gram of biomass. In some embodiments, the ratio of ozone to the lignocellulosic biomass may be from about 1:100 to about 1:50. Lower ratios may also be used. Preferred is a ratio of ozone to biomass which is sufficient to fragment lignin while retaining carbohydrate in good yield. Use of excess ozone beyond that which is optimal for delignification may lead to carbohydrate loss and lower sugar yields through saccharification.

### Ozone Treatment Conditions:

Contacting of the native lignocellulosic biomass or the ammonia-pretreated biomass with a gas comprising ozone may be carried out in any suitable vessel, such as a batch reactor or a continuous reactor. Typically the vessel is one that has a mechanism for heating or cooling, and has a mechanism for mixing the contents. Optionally, the vessel is one that can withstand pressure. The ozone pretreatment reaction may be performed in a fixed bed reactor, for example, or in a rotating horizontal cylinder, or a continuous stirred tank reactor. The suitable vessel may be equipped with a means, such as impellers, for agitating the biomass or the aqueous biomass suspension, or the vessel itself may rotate or spin to agitate the solid biomass. Reactor design is discussed in Lin, K.-H., and Van Ness, H. C. (in Perry, R. H. and Chilton, C. H. (eds), Chemical Engineer's Handbook, 5^{th} Edition (1973) Chapter 4, McGraw-Hill, NY). The ozone pretreatment reaction may be carried out as a batch process, or as a continuous process. The biomass may be contacted with ozone in the same reactor as the ammonia-treatment is performed, or in another reactor. The biomass may be contacted with ozone in one reactor, then saccharified in the same vessel; alternatively, saccharification may be performed in a separate vessel.

Prior to contacting the biomass with a gas comprising ozone, the native lignocellulosic biomass or the ammonia-pretreated biomass may be dried by conventional means. The dried native lignocellulosic or ammonia-pretreated biomass may contain about 10 percent to about 70 percent moisture, for example from about 30 percent to about 60 percent moisture.

Contacting the biomass with a gas comprising ozone may be carried out at a temperature of from about 0° C. to about 50° C. In one embodiment, the temperature may be from about 0° C. to about 25° C. Higher temperatures may be used but are generally less practical as ozone decomposition increases with increasing temperature. Lower temperatures may also be used but are generally less economical due to cooling requirements and, in the case where an aqueous biomass suspension is used, may not be practical from an operability standpoint. Contacting the biomass with a gas comprising ozone may be carried out for a reaction time of at least about 1 minute, for example for a reaction time of about 1 minute to about 60 minutes, or about 1 minute to about 30 minutes, or about 1 minute to about 25 minutes, or about 1 minute to about 20 minutes, or about 1 minute to about 15 minutes, or about 1 minute to about 10 minutes, or about 1 minute to about 5 minutes. Extending the ozonation time beyond that optimal for lignin degradation may result in decreased sugar yields, presumably due to sugar degradation.

Contacting the biomass with a gas comprising ozone may be performed at autogeneous pressure. Higher or lower pressures may also be used but are generally less practical.

The biomass may be contacted in the solid state with the gas comprising ozone, without a liquid phase being present. Alternatively, the biomass may be contacted as an aqueous suspension with the gas comprising ozone. To generate a biomass suspension, ammonia-treated biomass is contacted with an aqueous solution. The weight percent of biomass in the aqueous ammonia-treated biomass suspension can be from about 20 weight percent to about 70 weight percent, for example from about 30 weight percent to about 60 weight percent. The aqueous ammonia-treated biomass suspension can have a pH of about 1 to about 10, for example from about 2 to about 9, or from about 1 to about 7, or from about 1 to about 5. The aqueous solution may further comprise a buffer, for example a citrate buffer. The selection of an appropriate buffer may be based on the buffer's suitability for controlling pH in a subsequent saccharification. After ozone treatment is complete, the pH of the aqueous biomass suspension can be adjusted to a second pH sufficient for enzymatic saccharification of the biomass, if desired.

For the ozone pretreatment step, the temperature, time for pretreatment, ozone concentration in the gas, moisture content, biomass concentration, ratio of ozone to biomass, biomass type, and biomass particle size are related; thus these variables may be adjusted as necessary for each type of biomass to optimize the pretreatment processes described herein.

To assess performance of the pretreatment, i.e., the production of readily saccharifiable biomass, and subsequent saccharification, separately or together, the theoretical yield of sugars derivable from the starting biomass may be determined and compared to measured yields.

### Further Processing:

Saccharification:

Following pretreatments of ozone followed by aqueous ammonia, or aqueous ammonia followed by ozone, the readily saccharifiable biomass comprises a mixture of fragmented lignin and polysaccharides. If desired, prior to further processing, the lignin fragments or oxidation products may be removed from the pretreated biomass by filtering and optionally washing the sample with EtOH/H₂O (0% to 100% EtOH volume/volume [v/v]). As the filtration and washing steps are not necessary to obtain improved sugar yields, and as the costs associated with them may negatively impact the economics of the method, filtering and washing of the biomass is preferably omitted. The biomass may be dried at room temperature, resulting in readily saccharifiable biomass. The concentration of glucan, xylan and acid-insoluble lignin content of the readily saccharifiable biomass may be determined using analytical means well known in the art.

The readily saccharifiable biomass may then be further hydrolyzed in the presence of a saccharification enzyme consortium to release oligosaccharides and/or monosaccharides in a hydrolysate. Surfactants such as polyethylene glycols (PEG) may be added to improve the saccharification process (U.S. Pat. No. 7,354,743 B2, incorporated herein by reference). Saccharification enzymes and methods for biomass treatment are reviewed in Lynd, L. R., et al. (Microbiol. Mol. Biol. Rev., 66:506-577, 2002). The saccharification enzyme consortium may comprise one or more glycosidases; the glycosidases may be selected from the group consisting of cellulose-hydrolyzing glycosidases, hemicellulose-hydrolyzing glycosidases, and starch-hydrolyzing glycosidases. Other enzymes in the saccharification enzyme consortium may include peptidases, lipases, ligninases and feruloyl esterases.

The saccharification enzyme consortium comprises one or more enzymes selected primarily, but not exclusively, from the group “glycosidases” which hydrolyze the ether linkages of di-, oligo-, and polysaccharides and are found in the enzyme classification EC 3.2.1.x (Enzyme Nomenclature 1992, Academic Press, San Diego, Calif. with Supplement 1 (1993), Supplement 2 (1994), Supplement 3 (1995, Supplement 4 (1997) and Supplement 5 [in Eur. J. Biochem., 223:1-5, 1994; Eur. J. Biochem., 232:1-6, 1995; Eur. J. Biochem., 237:1-5, 1996; Eur. J. Biochem., 250:1-6, 1997; and Eur. J. Biochem., 264:610-650 1999, respectively]) of the general group “hydrolases” (EC 3). Glycosidases useful in the present method can be categorized by the biomass component that they hydrolyze. Glycosidases useful for the present method include cellulose-hydrolyzing glycosidases (for example, cellulases, endoglucanases, exoglucanases, cellobiohydrolases, β-glucosidases), hemicellulose-hydrolyzing glycosidases (for example, xylanases, endoxylanases, exoxylanases, β-xylosidases, arabino-xylanases, mannases, galactases, pectinases, glucuronidases), and starch-hydrolyzing glycosidases (for example, amylases, α-amylases, β-amylases, glucoamylases, α-glucosidases, isoamylases). In addition, it may be useful to add other activities to the saccharification enzyme consortium such as peptidases (EC 3.4.x.y), lipases (EC 3.1.1.x and 3.1.4.x), ligninases (EC 1.11.1.x), and feruloyl esterases (EC 3.1.1.73) to help release polysaccharides from other components of the biomass. It is well known in the art that microorganisms that produce polysaccharide-hydrolyzing enzymes often exhibit an activity, such as cellulose degradation, that is catalyzed by several enzymes or a group of enzymes having different substrate specificities. Thus, a “cellulase” from a microorganism may comprise a group of enzymes, all of which may contribute to the cellulose-degrading activity. Commercial or non-commercial enzyme preparations, such as cellulase, may comprise numerous enzymes depending on the purification scheme utilized to obtain the enzyme. Thus, the saccharification enzyme consortium of the present method may comprise enzyme activity, such as “cellulase”, however it is recognized that this activity may be catalyzed by more than one enzyme.

Saccharification enzymes may be obtained commercially, in isolated form, such as Spezyme® CP cellulase (Genencor International, Rochester, N.Y.) and Multifect® xylanase (Genencor). In addition, saccharification enzymes may be expressed in host organisms at the biofuels plant, including using recombinant microorganisms.

One skilled in the art would know how to determine the effective amount of enzymes to use in the consortium and adjust conditions for optimal enzyme activity. One skilled in the art would also know how to optimize the classes of enzyme activities required within the consortium to obtain optimal saccharification of a given pretreatment product under the selected conditions.

Preferably the saccharification reaction is performed at or near the temperature and pH optima for the saccharification enzymes. The temperature optimum used with the saccharification enzyme consortium in the present method ranges from about 15° C. to about 100° C. In another embodiment, the temperature optimum ranges from about 20° C. to about 80° C. Most typically the temperature optimum ranges from about 45° C. to about 50° C. The pH optimum can range from about 2 to about 11. In another embodiment, the pH optimum used with the saccharification enzyme consortium in the present method may range from about 4 to about 5.5.

The saccharification may be performed for a time of about several minutes to about 120 hours, and preferably from about several minutes to about 48 hours. The time for the reaction will depend on enzyme concentration and specific activity, as well as the substrate used and the environmental conditions, such as temperature and pH. One skilled in the art can readily determine optimal conditions of temperature, pH and time to be used with a particular substrate and saccharification enzyme(s) consortium.

The saccharification may be performed batch-wise or as a continuous process. The saccharification may also be performed in one step, or in a number of steps. For example, different enzymes required for saccharification may exhibit different pH or temperature optima. A primary treatment may be performed with enzyme(s) at one temperature and pH, followed by secondary or tertiary (or more) treatments with different enzyme(s) at different temperatures and/or pH. In addition, treatment with different enzymes in sequential steps may be at the same pH and/or temperature, or different pHs and temperatures, such as using hemicellulases stable and more active at higher pHs and temperatures followed by cellulases that are active at lower pHs and temperatures.

The degree of solubilization of sugars from biomass following saccharification may be monitored by measuring the release of monosaccharides and oligosaccharides. Methods to measure monosaccharides and oligosaccharides are well known in the art. For example, the concentration of reducing sugars can be determined using the 1,3-dinitrosalicylic (DNS) acid assay (Miller, G. L., Anal. Chem., 31: 426-428, 1959). Alternatively, sugars can be measured by HPLC using an appropriate column as described below.

### Fermentation to Target Products:

The readily saccharifiable biomass produced by the present methods may be hydrolyzed by enzymes as described above to produce fermentable sugars which then can be fermented into a target product. “Fermentation” refers to any fermentation process or any process comprising a fermentation step. Target products include, without limitation alcohols (e.g., arabinitol, butanol, ethanol, glycerol, methanol, 1,3-propanediol, sorbitol, and xylitol); organic acids (e.g., acetic acid, acetonic acid, adipic acid, ascorbic acid, citric acid, 2,5-diketo-D-gluconic acid, formic acid, fumaric acid, glucaric acid, gluconic acid, glucuronic acid, glutaric acid, 3-hydroxypropionic acid, itaconic acid, lactic acid, malic acid, malonic acid, oxalic acid, propionic acid, succinic acid, and xylonic acid); ketones (e.g., acetone); amino acids (e.g., aspartic acid, glutamic acid, glycine, lysine, serine, and threonine); gases (e.g., methane, hydrogen (H₂), carbon dioxide (CO₂), and carbon monoxide (CO)).

Fermentation processes also include processes used in the consumable alcohol industry (e.g., beer and wine), dairy industry (e.g., fermented dairy products), leather industry, and tobacco industry.

Further to the above, the sugars produced from saccharifying the pretreated biomass as described herein may be used to produce in general, organic products, chemicals, fuels, commodity and specialty chemicals such as xylose, acetone, acetate, glycine, lysine, organic acids (e.g., lactic acid), 1,3-propanediol, butanediol, glycerol, ethylene glycol, furfural, polyhydroxyalkanoates, cis, cis-muconic acid, and animal feed (Lynd, L. R., Wyman, C. E., and Gerngross, T. U., Biocommodity Engineering, Biotechnol. Prog., 15: 777-793, 1999; and Philippidis, G. P., Cellulose bioconversion technology, in Handbook on Bioethanol: Production and Utilization, Wyman, C. E., ed., Taylor & Francis, Washington, D.C., 179-212, 1996; and Ryu, D. D. Y., and Mandels, M., Cellulases: biosynthesis and applications, Enz. Microb. Technol., 2: 91-102, 1980).

Potential coproducts may also be produced, such as multiple organic products from fermentable carbohydrate. Lignin-rich residues remaining after pretreatment and fermentation can be converted to lignin-derived chemicals, chemical building blocks or used for power production.

Conventional methods of fermentation and/or saccharification are known in the art including, but not limited to, saccharification, fermentation, separate hydrolysis and fermentation (SHF), simultaneous saccharification and fermentation (SSF), simultaneous saccharification and cofermentation (SSCF), hybrid hydrolysis and fermentation (HHF), and direct microbial conversion (DMC).

SHF uses separate process steps to first enzymatically hydrolyze cellulose to sugars such as glucose and xylose and then ferment the sugars to ethanol. In SSF, the enzymatic hydrolysis of cellulose and the fermentation of glucose to ethanol is combined in one step (Philippidis, G. P., in Handbook on Bioethanol: Production and Utilization, Wyman, C. E., ed., Taylor & Francis, Washington, D.C., 179-212, 1996). SSCF includes the cofermentation of multiple sugars (Sheehan, J., and Himmel, M., Bioethanol, Biotechnol. Prog. 15: 817-827, 1999). HHF includes two separate steps carried out in the same reactor but at different temperatures, i.e., high temperature enzymatic saccharification followed by SSF at a lower temperature that the fermentation strain can tolerate. DMC combines all three processes (cellulase production, cellulose hydrolysis, and fermentation) in one step (Lynd, L. R., Weimer, P. J., van Zyl, W. H., and Pretorius, I. S., Microbiol. Mol. Biol. Reviews, 66: 506-577, 2002).

These processes may be used to produce target products from the readily saccharifiable biomass produced by the pretreatment methods described herein.

### Advantages of the Present Methods:

One of the advantages of the present methods is the high selectivity for fragmenting and removing lignin from the biomass while leaving the carbohydrates largely intact. Less selective pretreatment methods hydrolyze a portion of the carbohydrates to sugars, for example a portion of the glucans to glucose and/or a portion of the xylans to xylose. If present, the monomeric sugars can be degraded during the pretreatment process, resulting in a decrease in the overall yield to sugar (i.e. through a saccharification step). As demonstrated by the Examples, prolonged ozonation can lead to diminished yields of sugars, in particular xylose. Therefore, there exists an optimal reaction time for ozone treatment, below which the pretreatment will be ineffective, and above which it will be unselective. The optimal reaction time for ozone treatment depends in part on the biomass composition, in particular lignin content, the particle size, and the amount of ozone used relative to the biomass.

Another advantage of the present methods is that separation or washing of the biomass after ozone treatment to physically remove the oxidized and fragmented lignin is not necessary. The monomeric sugars, being more soluble than cellulose and hemicellulose, can be separated from the carbohydrates when filtration and washing of the treated biomass are necessary before saccharification, resulting in a decrease in the overall yield to sugar. The present methods minimize sugar loss during lignin oxidation and fragmentation, which is of economic benefit.

In particular, the present methods provide surprisingly good xylose recovery through saccharification. Xylose recovery can be substantially lower than glucose recovery, when compared to the theoretical yields of the sugars based on the total amount of sugars present in the native biomass before any pretreatment. This arises from the vast difference in the kinetics of hydrolysis of xylans and glucans, which are more difficult and easier to hydrolyze, respectively. It was not expected that xylose recovery would be as high as seen with the present methods using optimal reaction conditions. Upon ozone treatment, the lignin, hemicellulose, and cellulose content of the biomass is decreased, with lignin being the most severely affected, followed by hemicellulose and cellulose, respectively. The present methods provide conditions under which lignin is selectively degraded in the presence of hemicellulose and cellulose, without negatively affecting their saccharification yields. This is especially significant in the case of xylose yield, as hemicellulose is more easily degraded with ozone.

Additionally, lignin is more electron rich than the carbohydrates contained in biomass, and as a result the lignin is more prone to oxidation by the ozone than are the carbohydrates. While not wishing to be bound by any theory, oxidation of the lignin by the ozone is believed to reduce the molecular weight of the lignin fragments, which in turn renders the lignin fragments both more soluble in the solvent solution and less able to bind to cellulolytic enzymes. As a result, the use of lower enzyme loadings in saccharification is enabled, which can provide cost savings with regard to enzyme usage. The present methods advantageously combine the use of pretreatment with an aqueous solution comprising ammonia followed by selective oxidation of lignin by ozone treatment to produce a readily saccharifiable biomass.

The present methods offer advantageous flexibility regarding ozonation in that the ozone treatment may be performed on solid biomass or on an aqueous suspension of biomass. Both options offer opportunity for overall process simplification and economic benefit. For example, if desired the biomass may be treated with ozone as an aqueous biomass suspension, wherein the suspension is formed from an aqueous solution comprising a buffer selected for a subsequent saccharification step. After ozone treatment, the enzyme cocktail may be added directly to the readily saccharifiable biomass and saccharification can be performed in the same reaction vessel. Alternatively, solid biomass may be contacted with ozone, that is, without the presence of a liquid phase.

### EXAMPLES

The goal of the experimental work described below was to develop a pretreatment process for lignocellulose that maximized lignin degradation and minimized carbohydrate loss in the pretreatment to produce a readily saccharifiable biomass that may be further processed to result in a maximal monomeric sugar yield following enzymatic saccharification. The approach adopted was to selectively oxidize and fragment the lignin in the presence of a gas comprising ozone while retaining the sugars with the solids residue. The following experiments show that ozone treatment of ammonia-pretreated biomass oxidized and fragmented the lignin to produce a readily saccharifiable biomass.

The present invention is further defined in the following examples. It should be understood that these examples, while indicating preferred embodiments of the invention, are given by way of illustration only. From the above discussion and these examples, one skilled in the art can ascertain the essential characteristics of this invention, and without departing from the spirit and scope thereof, can make various changes and modifications of the invention to adapt it to various uses and conditions.

The following materials were used in the examples. All commercial reagents were used as received.

Glucose, xylose, cellobiose, and citric acid were obtained from Sigma-Aldrich (St. Louis, Mo.). Suitable zirconium pellets can be obtained from Union Process (Akron, Ohio) or Ortech Advanced Ceramics (Sacramento, Calif.).

Corn cob was obtained from University of Wisconsin Farm, in Madison, Wis. and was milled to assorted sizes. Switchgrass was obtained from Genera Energy. The switchgrass sample particles were less than 1 mm in size, and the initial moisture content was about 7 weight percent.

### Carbohydrate Analysis of Biomass

A modified version of the NREL LAP procedure “Determination of Structural Carbohydrates and Lignin in Biomass” was used to determine the weight percent glucan and xylan in the biomass. Sample preparation was simplified by drying at 80° C. under vacuum or at 105° C. under ambient pressure overnight. The samples were knife milled to pass through a 20 mesh screen but were not sieved. The dry milled solids were than subjected to the acid hydrolysis procedure at a 50 mg solids scale. The solids were not first extracted with water or ethanol. HPLC analysis of sugars was done on an Aminex HPX-87H column and no analysis of lignin was attempted.

The soluble sugars glucose, cellobiose, and xylose in saccharification liquor were measured by HPLC (Agilent 1100, Santa Clara, Calif.) using Bio-Rad HPX-87H column (Bio-Rad Laboratories, Hercules, Calif.) with appropriate guard columns, using 0.01 N aqueous sulfuric acid as the eluant. The sample pH was measured and adjusted to 5-6 with sulfuric acid if necessary. The sample was then passed through a 0.2 μm syringe filter directly into an HPLC vial. The HPLC run conditions were as follows:
Biorad Aminex HPX-87H (for carbohydrates):
Injection volume: 10-50 μL, dependent on concentration and detector limits
Mobile phase: 0.01 N aqueous sulfuric acid, 0.2 micron filtered and degassed
Flow rate: 0.6 mL/minute
Column temperature: 50° C., guard column temperature <60° C.
Detector temperature: as close to main column temperature as possible
Detector: refractive index
Run time: 15 minute data collection
   After the run, concentrations in the sample were determined from standard curves for each of the compounds.

Ozone was generated from air using an ozonizer (model CD1500) manufactured by ClearWater Tech (San Luis Obispo, Calif.) and set on maximum voltage. The amount of ozone used in each Example with an ozonation step was calculated from the ozone consumed during the indicated reaction time by measuring the ozone concentration in the ozone-enriched air entering and leaving the experimental apparatus and taking the difference. Ozone measurements were made using a Teledyne Instruments (San Diego, Calif.) ozone monitor, model 450 M.

The moisture content of the biomass was determined by the National Renewable Energy Laboratory (NREL) procedure “Determination of Total Solids in Biomass and Total Dissolved Solids in Liquid Process Samples”.

The roll mill was manufactured by US Stoneware (East Palestine, Ohio).

The following abbreviations are used:

“HPLC” is High Performance Liquid Chromatography, “C” is degrees Centigrade or Celsius; “%” is percent; “mL” is milliliter; “h” is hour(s); “rpm” is revolution per minute; “EtOH” is ethanol; “mg/g” is milligram per gram; “g/100 mL” is gram per 100 milliliter; “g” is gram; “NaOH” is sodium hydroxide; “w/v” is weight per volume; “v/v” is volume for volume, “w/w” is weight for weight; “mm” is millimeter; “mL/min” is milliliter per minute; “min” is minutes; “mM” is millimolar, “N” is normal, “μL” is microliter.

The biomass used in Examples 1-6 and Comparative Examples A and B was pretreated with an aqueous solution comprising ammonia according to the following procedure. The same procedure was used in Examples 7-15 and Comparative Examples C through H, except that switchgrass was used in place of corn cob and ammonia treatment was the second or only pretreatment step.

To a 170 L jacketed horizontal paddle reactor (Jaygo Manufacturing, Inc, Mahwah, N.J.) were added corn cobs (25.7 kg). Vacuum was then applied to the reactor to reach a pressure of −10.5 psig. After that, an aqueous solution of ammonia (14.88 kg of 7.63 wt % of ammonia) was added to the reactor, followed by water (1.58 kg). Steam was then injected to bring the reactor temperature to 145° C. for 20 minutes. The reactor was then vented to atmospheric pressure, and the mixture was then evacuated under vacuum to a pressure of −10.5 psig. Sterile air was then introduced to the reactor, the pretreated cob was collected and ground using a hammer mill, and sieved through a ½ inch screen.

Examples 1 through 3 and Examples 4 through 6 illustrate a method for producing readily saccharifiable biomass by pretreatment with dilute ammonia followed by ozonation of the biomass as an aqueous suspension. The effect of the pretreatment was quantified by saccharifying the readily saccharifiable biomass to determine the theoretical yields for glucose and xylose. Theoretical yields encompass the monomeric sugar yields obtained through the pretreatment and saccharification steps compared to the total amount of sugars present in the native biomass before any pretreatment was performed. Biomass samples from Examples 1-3 were saccharified using Spezyme, Novo 188, and Multifect enzyme cocktails; Examples 4-6 were saccharified at lower enzyme loading, and with Accellerase/Multifect enzyme cocktails.

To demonstrate the beneficial effect of the ozonation, Comparative Example A was performed as a control experiment following the same procedure as Examples 1-3 but without the ozone treatment step. The ammonia-pretreated biomass of Comparative Example A was saccharified using Spezyme, Novo 188, and Multifect enzyme cocktails. As another control experiment, Comparative Example B was performed following the same procedure as Examples 4-6 but without the ozone treatment step. The ammonia-pretreated biomass of Comparative Example B was saccharified at lower enzyme loading and with Accellerase/Multifect enzyme cocktails.

For Examples 1-3 and Comparative Example A, theoretical yields for glucose and xylose are given in Tables 1 and 2, respectively. For Examples 4-6 and Comparative Example B, theoretical yields for glucose and xylose are given in Tables 3 and 4, respectively.

### Example 1

### Effect of Ozonation of an Aqueous Ammonia-Treated Biomass Suspension for 10 Minutes

To a slurry of ammonia-pretreated corn cob (6.0 g of 60% dry solid, 3.6 g dry solid) in citrate buffer (19.36 mL, pH=5) was introduced a stream of ozone-enriched air (flow rate 2 L/min) at room temperature. After 10 minutes and the consumption of 5.0 mg of ozone, the flow of ozone-enriched air was stopped and the slurry was charged with Spezyme (150.0 μL, concentration 168.5 mg/mL), Multifect (180.0 μL, concentration 56.1 mg/mL), and Novozyme 188 (25.0 μL, concentration 253 mg/mL) enzyme cocktails, and the mixture was left stirring in an incubator/shaker at 48° C. Samples were taken every 24 h and analyzed by HPLC to determine the monomeric sugar yields versus time. Results are shown in Tables 1 and 2.

### Example 2

### Effect of Ozonation of an Aqueous Ammonia-Treated Biomass Suspension for 20 Minutes

To a slurry of ammonia-pretreated corn cob (6.0 g of 60% dry solid, 3.6 g dry solid) in citrate buffer (19.36 mL, pH=5) was introduced a stream of ozone-enriched air (flow rate 2 L/min) at room temperature. After 20 minutes and the consumption of 10.0 mg of ozone, the flow of ozone-enriched air was stopped and the slurry was charged with Spezyme (150.0 μL, concentration 168.5 mg/mL), Multifect (180.0 μL, concentration 56.1 mg/mL), and Novozyme 188 (25.0 μL, concentration 253 mg/mL) enzyme cocktails, and the mixture was left stirring in an incubator/shaker at 48° C. Samples were taken every 24 h and analyzed by HPLC to determine the monomeric sugar yields versus time. Results are shown in Tables 1 and 2.

### Example 3

### Effect of Ozonation of an Aqueous Ammonia-Treated Biomass Suspension for 30 Minutes

To a slurry of ammonia-pretreated corn cob (6.0 g of 60% dry solid, 3.6 g dry solid) in citrate buffer (19.36 mL, pH=5) was introduced a stream of ozone-enriched air (flow rate 2 L/min) at room temperature. After 30 minutes and the consumption of 15.1 mg of ozone, the flow of ozone-enriched air was stopped and the slurry was charged with Spezyme (150.0 μL, concentration 168.5 mg/mL), Multifect (180.0 μL, concentration 56.1 mg/mL), and Novozyme 188 (25.0 μL, concentration 253 mg/mL) enzyme cocktails, and the mixture was left stirring in an incubator/shaker at 48° C. Samples were taken every 24 h and analyzed by HPLC to determine the monomeric sugar yields versus time. Results are shown in Tables 1 and 2.

### Comparative Example A

### Control Experiment with No Ozonation

To a slurry of ammonia-pretreated corn cob (6.0 g of 60% dry solid, 3.6 g dry solid) in citrate buffer (19.36 mL, pH=5) was added Spezyme (150.0 μL, concentration 168.5 mg/mL), Multifect (180.0 μL, concentration 56.1 mg/mL), and Novozyme 188 (25.0 μL, concentration 253.0 mg/mL) enzyme cocktails, and the mixture was left stirring in an incubator/shaker at 48° C. Samples were taken every 24 h and analyzed by HPLC to determine the monomeric sugar yields versus time. Results are shown in Tables 1 and 2.

TABLE 1
Theoretical yields for glucose during saccharification
of biomass from Examples 1-3 and Comparative Example A.

| Saccharification | Comparative | | | |
|---|---|---|---|---|
| Time (hours) | Example A | Example 1 | Example 2 | Example 3 |
| 0 | 0 | 0 | 0 | 0 |
| 24 | 32.1 | 39.9 | 38.8 | 42.2 |
| 48 | 35.1 | 51.2 | 53.4 | 47.9 |
| 72 | 39.3 | 58.1 | 55.6 | 49.3 |
| 96 | 41.3 | 66.1 | 56.2 | 49.6 |
| 120 | 45.1 | 68.7 | 63.5 | 51.5 |

TABLE 2
Theoretical yields for xylose during saccharification
of biomass from Examples 1-3 and Comparative Example A.

| Saccharification | Comparative | | | |
|---|---|---|---|---|
| Time (hours) | Example A | Example 1 | Example 2 | Example 3 |
| 0 | 0 | 0 | 0 | 0 |
| 24 | 24.7 | 32.9 | 19.1 | 29.7 |
| 48 | 29.4 | 40.5 | 25.2 | 31.8 |
| 72 | 34.0 | 42.0 | 26.1 | 33.0 |
| 96 | 44.8 | 50.4 | 26.4 | 33.8 |
| 120 | 45.4 | 50.8 | 30.3 | 36.9 |

As shown in Table 1, ozone treatment of the ammonia-treated corn cob provided readily saccharifiable biomass which provided improved glucose yields upon saccharification. Higher glucose yields were observed with longer ozone treatment reaction times. The data in Table 2 indicates that there was an optimal reaction time for ozone treatment and xylose recovery. With longer reaction times for ozone treatment, xylose was degraded and xylose yield dropped.

### Example 4

### Effect of Ozonation of an Aqueous Ammonia-Treated Biomass Suspension for 10 Minutes and Saccharification at Lower Enzyme Loading

To a slurry of dilute ammonia-pretreated corn cob (4.24 g of dry solid) in citrate buffer (26.6 mL, pH=5) was introduced a stream of ozone-enriched air (flow rate 2 L/min) at room temperature. After 10 minutes and the consumption of 5.0 mg of ozone, the flow of ozone-enriched air was stopped and the slurry was charged with Accellerase® 1000 (393.0 μL, concentration 97.1 mg/mL) and Multifect CX 12L (180.0 μL, concentration 56.1 mg/mL) enzyme cocktails, and the mixture was left stirring in an incubator/shaker at 48° C. Samples were taken every 24 h and analyzed by HPLC to determine the monomeric sugar yields versus time. Results are shown in Tables 3 and 4.

### Example 5

### Effect of Ozonation of an Aqueous Ammonia-Treated Biomass Suspension for 20 Minutes and Saccharification at Lower Enzyme Loading

To a slurry of dilute ammonia-pretreated corn cob (4.24 g of dry solid) in citrate buffer (26.6 mL, pH=5) was introduced a stream of ozone-enriched air (flow rate 2 L/min) at room temperature. After 20 minutes and the consumption of 10.0 mg of ozone, the flow of ozone-enriched air was stopped and the slurry was charged with Accellerase® 1000 (393.0 μL, concentration 97.1 mg/mL) and Multifect CX 12L (180.0 μL, concentration 56.1 mg/mL) enzyme cocktails, and the mixture was left stirring in an incubator/shaker at 48° C. Samples were taken every 24 h and analyzed by HPLC to determine the monomeric sugar yields versus time. Results are shown in Tables 3 and 4.

### Example 6

### Effect of Ozonation of an Aqueous Ammonia-Treated Biomass Suspension for 30 Minutes and Saccharification at Lower Enzyme Loading

To a slurry of dilute ammonia-pretreated corn cob (4.24 g of dry solid) in citrate buffer (26.6 mL, pH=5) was introduced a stream of ozone-enriched air (flow rate 2 L/min) at room temperature. After 30 minutes and the consumption of 15.1 mg of ozone, the flow of ozone-enriched air was stopped and the slurry was charged with Accellerase® 1000 (393.0 μL, concentration 97.1 mg/mL) and Multifect CX 12L (180.0 μL, concentration 56.1 mg/mL) enzyme cocktails, and the mixture was left stirring in an incubator/shaker at 48° C. Samples were taken every 24 h and analyzed by HPLC to determine the monomeric sugar yields versus time. Results are shown in Tables 3 and 4.

### Comparative Example B

### Control Experiment with No Ozonation and Saccharification at Lower Enzyme Loading

To a slurry of ammonia-pretreated corn cob (4.24 g dry solid) in citrate buffer (26.6 mL, pH=5) was charged Accellerase® 1000 (393.0 μL, concentration 97.1 mg/mL) and Multifect CX 12L (180.0 μL, concentration 56.1 mg/mL) enzyme cocktails, and the mixture was left stirring in an incubator/shaker at 48° C. Samples were taken every 24 h and analyzed by HPLC to determine the monomeric sugar yields versus time. Results are shown in Tables 3 and 4.

TABLE 3
Theoretical yields for glucose during saccharification
of biomass from Examples 4-6 and Comparative Example B.

| Saccharification | Comparative | | | |
|---|---|---|---|---|
| Time (hours) | Example B | Example 4 | Example 5 | Example 6 |
| 0 | 0 | 0 | 0 | 0 |
| 24 | 25.5 | 31.2 | 32.2 | 31.5 |
| 48 | 35.8 | 43.2 | 44.1 | 50.0 |
| 72 | 39.7 | 48.3 | 51.1 | 51.2 |
| 96 | 39.9 | 52.8 | 56.9 | 58.1 |
| 120 | 48.6 | 61.0 | 61.9 | 65.2 |

Enzyme loadings: 9 mg/g dry solid Accellerase ®, 3 mg/g dry solid Multifect.

TABLE 4
Theoretical yields for xylose during saccharification
of biomass from Examples 5-7 and Comparative Example B.

| Saccharification | Comparative | | | |
|---|---|---|---|---|
| Time (hours) | Example B | Example 4 | Example 5 | Example 6 |
| 0 | 0 | 0 | 0 | 0 |
| 24 | 19.8 | 22.5 | 24.3 | 24.0 |
| 48 | 26.6 | 32.0 | 32.8 | 36.4 |
| 72 | 29.2 | 35.7 | 37.2 | 36.7 |
| 96 | 29.2 | 38.2 | 40.5 | 40.3 |
| 120 | 35.3 | 43.8 | 43.3 | 44.7 |

Enzyme loadings: 9 mg/g dry solid Accellerase ®, 3 mg/g dry solid Multifect.

The results demonstrate that ozone treatment of the biomass provided a readily saccharifiable biomass and resulted in higher theoretical yields for both glucose and xylose.

Ozone treatment of wet solid biomass resulted in increased yields of both glucose and xylose sugars.

Examples 7 through 15 illustrate a method for producing readily saccharifiable biomass by pretreatment with a gas comprising ozone followed by contacting with an aqueous solution comprising ammonia. The effect of the pretreatment was quantified by saccharifying the readily saccharifiable biomass to determine the theoretical yields for glucose and xylose. Theoretical yields encompass the monomeric sugar yields obtained through the pretreatment and saccharification steps compared to the total amount of sugars present in the native biomass before any pretreatment was performed. Pretreated biomass samples from Examples 7 through 15 were saccharified using Accellerase® 1500 and other saccharification enzymes.

To demonstrate the beneficial effect of the ozonation, Comparative Examples C, D, and E were performed as control experiments following the same procedure as Examples 7, 8, and 9 but without the ozone treatment step. As other control experiments, Comparative Examples F, G, and H were performed following the same procedure as Examples 10, 11, and 12 but without the ozone treatment step. Biomass samples from Comparative Examples C through H were saccharified using Accellerase® 1500 and other saccharification enzymes.

For Examples 7, 8, and 9 and Comparative Examples C, D, and E, theoretical yield for glucose and xylose are given in Tables 5 and 6, respectively. For Examples 10, 11, and 12 and Comparative Examples F, G, and H, theoretical yields for glucose and xylose are given in Tables 7 and 8, respectively. For Examples 13, 14, and 15, theoretical yields for glucose and xylose are given in Tables 9 and 10, respectively, along with results for Comparative Examples C, D, and E.

### Example 7

### Effect of Ozonation of Biomass Followed by Ammonia Treatment for 20 Minutes at 145° C.

Switchgrass (7.0 g dry material, adjusted to 60% moisture by addition of water) was placed in a 250 mL bottle and 5 mm Zirconium pellets (30 g) were added. The bottle was placed on a roll mill and spun at 100 rpm for 90 minutes. Simultaneously, a stream of ozone-enriched air was introduced to the bottle; during the reaction time 75.3 mg of O₃ was consumed. 500 Milligrams of the ozone-treated biomass was then transferred to a pressure vessel and mixed with aqueous ammonium hydroxide (0.107 mL of 28% NH₃ in water), and 0.643 mL of water. The mixture was heated for 20 minutes at 145° C. Upon cooling, the resulting material was dried in vacuo, and 252 mg were suspended in pH 5 buffer (1.500 mL, 14% solids loading). To the resulting slurry Accellerase® 1500 (20.4 μL, 118 mg/mL) and other saccharification enzymes were added, and the mixture was left stirring in an incubator/shaker at 48° C. Samples were taken every 24 h and analyzed by HPLC to generate data on monomeric sugar yields versus time. Results are shown in Tables 5 and 6.

### Example 8

### Effect of Ozonation of Biomass Followed by Ammonia Treatment for 60 Minutes at 145° C.

Switchgrass (7.0 g dry material, adjusted to 60% moisture by addition of water) was placed in a 250 mL bottle and 5 mm Zirconium pellets (30 g) were added. The bottle was placed on a roll mill and spun at 100 rpm for 90 minutes. Simultaneously, a stream of ozone-enriched air was introduced to the bottle; during the reaction time 75.3 mg of O₃ was consumed. 500 Milligrams of the ozone-treated biomass was then transferred to a pressure vessel and mixed with aqueous ammonium hydroxide (0.107 mL of 28% NH₃ in water) and 0.643 mL of water. The mixture was heated for 60 minutes at 145° C. Upon cooling, the resulting material was dried in vacuo, and 253 mg were suspended in pH 5 buffer (1.500 mL, 14% solids loading). To the resulting slurry Accellerase® 1500 (20.4 μL, 118 mg/mL) and other saccharification enzymes were added, and the mixture was left stirring in an incubator/shaker at 48° C. Samples were taken every 24 h and analyzed by HPLC to generate data on monomeric sugar yields versus time. Results are shown in Tables 5 and 6.

### Example 9

### Effect of Ozonation of Biomass Followed by Ammonia Treatment for 20 Minutes at 145° C.

Switchgrass (7.0 g dry material, adjusted to 60% moisture by addition of water) was placed in a 250 mL bottle and 5 mm Zirconium pellets (30 g) were added. The bottle was placed on a roll mill and spun at 100 rpm for 90 minutes. Simultaneously, a stream of ozone-enriched air was introduced to the bottle; during the reaction time 75.3 mg O₃ was consumed. 500 Milligrams of the ozone-treated biomass was then transferred to a pressure vessel and mixed with aqueous ammonium hydroxide (0.107 mL of 28% NH₃ in water) and 0.643 mL of water. The mixture was heated for 90 minutes at 145° C. Upon cooling, the resulting material was dried in vacuo, and 251 mg were suspended in pH 5 buffer (1.500 mL, 14% solids loading). To the resulting slurry Accellerase® 1500 (20.4 μL, 118 mg/mL) and other saccharification enzymes were added, and the mixture was left stirring in an incubator/shaker at 48° C. Samples were taken every 24 h and analyzed by HPLC to generate data on monomeric sugar yields versus time. Results are shown in Tables 5 and 6.

### Comparative Example C

### Control Experiment: Ammonia Treatment for 20 Minutes at 145° C. (No Ozonation)

Switchgrass (500 mg) was placed into a pressure vessel and mixed with aqueous ammonium hydroxide (0.107 mL of 28% NH₃ in water) and 0.643 mL of water. The mixture was heated for 20 minutes at 145° C. Upon cooling, the resulting material was dried in vacuo, and 250 mg were suspended in pH 5 buffer (1.500 mL, 14% solids loading). To the resulting slurry Accellerase® 1500 (20.4 μL, 118 mg/mL) and other saccharification enzymes were added, and the mixture was left stirring in an incubator/shaker at 48° C. Samples were taken every 24 h and analyzed by HPLC to generate data on monomeric sugar yields versus time. Results are shown in Tables 5 and 6.

### Comparative Example D

### Control Experiment: Ammonia Treatment for 60 Minutes at 145° C. (No Ozonation)

Switchgrass (500 mg) was placed into a pressure vessel, and mixed with aqueous ammonium hydroxide (0.107 mL of 28% NH₃ in water) and 0.643 mL of water. The mixture was heated for 60 minutes at 145° C. Upon cooling, the resulting material was dried in vacuo, and 251 mg were suspended in pH 5 buffer (1.500 mL, 14% solids loading). To the resulting slurry Accellerase® 1500 (20.2 μL, 118 mg/mL) and other saccharification enzymes were added, and the mixture was left stirring in an incubator/shaker at 48° C. Samples were taken every 24 h and analyzed by HPLC to generate data on monomeric sugar yields versus time. Results are shown in Tables 5 and 6.

### Comparative Example E

### Control Experiment: Ammonia Treatment for 90 Minutes at 145° C. (No Ozonation)

Switchgrass (500 mg) was placed into a pressure vessel and mixed with aqueous ammonium hydroxide (0.107 mL of 28% NH₃ in water) and 0.643 mL of water. The mixture was heated for 90 minutes at 145° C. Upon cooling, the resulting material was dried in vacuo, and 252 mg were suspended in pH 5 buffer (1.500 mL, 14% solids loading). To the resulting slurry Accellerase® 1500 (20.4 μL, 118 mg/mL) and other saccharification enzymes were added, and the mixture was left stirring in an incubator/shaker at 48° C. Samples were taken every 24 h and analyzed by HPLC to generate data on monomeric sugar yields versus time. Results are shown in Tables 5 and 6

TABLE 5
Theoretical yields for glucose during saccharification of biomass
from Examples 7, 8, and 9 and Comparative Examples C, D, and E.

| Sacch. | Example | Example | Example | Comp. | Comp. | Comp. |
|---|---|---|---|---|---|---|
| Time (h) | 7 | 8 | 9 | Ex. C | Ex. D | Ex. E |
| 24 | 35.9 | 36.5 | 37.3 | 20.1 | 20.1 | 24.4 |
| 48 | 39.1 | 39.2 | 41.0 | 23.3 | 23.3 | 29.3 |
| 72 | 42.2 | 41.7 | 42.7 | 24.3 | 24.3 | 31.7 |
| 96 | 41.3 | 41.8 | 45.5 | 26.7 | 28.5 | 33.7 |
| 120 | 41.7 | 41.0 | 45.8 | 26.4 | 29.5 | 35.0 |
| 144 | 41.6 | 41.7 | 46.0 | 26.6 | 29.7 | 35.2 |

TABLE 6
Theoretical yields for xylose during saccharification of biomass
from Examples 7, 8, and 9 and Comparative Examples C, D, and E.

| Sacch. | Example | Example | Example | Comp. | Comp. | Comp. |
|---|---|---|---|---|---|---|
| Time (h) | 7 | 8 | 9 | Ex. C | Ex. D | Ex. E |
| 24 | 24.3 | 27.2 | 26.9 | 13.1 | 13.1 | 20.7 |
| 48 | 26.1 | 28.5 | 28.8 | 16.0 | 16.0 | 24.6 |
| 72 | 28.2 | 30.4 | 29.9 | 17.2 | 17.2 | 26.6 |
| 96 | 27.7 | 30.2 | 31.7 | 19.2 | 21.3 | 28.1 |
| 120 | 27.9 | 29.7 | 32.0 | 19.0 | 21.9 | 29.2 |
| 144 | 28.0 | 30.2 | 32.1 | 19.5 | 22.2 | 29.2 |

### Example 10

### Effect of Ozone Treatment Followed by Ammonia Treatment

Switchgrass (7.0 g dry material, adjusted to 60% moisture by addition of water) was placed in a 250 mL bottle and 5 mm Zirconium pellets (30 g) were added. The bottle was placed on a roll mill and spun at 100 rpm for 90 minutes. Simultaneously, a stream of ozone-enriched air was introduced to the bottle; during the reaction time 75.3 mg O₃ was consumed. 500 Milligrams of the ozone-treated biomass was then transferred to a pressure vessel and mixed with aqueous ammonium hydroxide (0.107 mL of 28% NH₃ in water) and 0.643 mL of water. The mixture was heated for 20 minutes at 155° C. Upon cooling, the resulting material was dried in vacuo, and 246 mg were suspended in pH 5 buffer (1.500 mL, 14% solids loading). To the resulting slurry Accellerase® 1500 (19.9 μL, 118 mg/mL) and other saccharification enzymes were added, and the mixture was left stirring in an incubator/shaker at 48° C. Samples were taken every 24 h and analyzed by HPLC to generate data on monomeric sugar yields versus time. Results are shown in Tables 7 and 8.

### Example 11

### Effect of Ozonation of Biomass Followed by Ammonia Treatment for 60 Minutes at 155° C.

Switchgrass (7.0 g dry material, adjusted to 60% moisture by addition of water) was placed in a 250 mL bottle and 5 mm Zirconium pellets (30 g) were added. The bottle was placed on a roll mill and spun at 100 rpm. Simultaneously, a stream of ozone-enriched air was introduced to the bottle; during the reaction time 75.3 mg O₃ was consumed. 500 Milligrams of this material was then transferred to a pressure vessel, and mixed with aqueous ammonium hydroxide (0.107 mL of 28% NH₃ in water) and 0.643 mL of water. The mixture was heated for 60 minutes at 155° C. Upon cooling, the resulting material was dried in vacuo, and 257 mg were suspended in pH 5 buffer (1.500 mL, 14% solids loading). To the resulting slurry Accellerase® 1500 (20.8 μL, 118 mg/mL) and other saccharification enzymes were added, and the mixture was left stirring in an incubator/shaker at 48° C. Samples were taken every 24 h and analyzed by HPLC to generate data on monomeric sugar yields versus time. Results are shown in Tables 7 and 8.

### Example 12

### Effect of Ozonation of Biomass Followed by Ammonia Treatment for 90 Minutes at 155° C.

Switchgrass (7.0 g dry material, adjusted to 60% moisture by addition of water) was placed in a 250 mL bottle and 5 mm Zirconium pellets (30 g) were added. The bottle was placed on a roll mill and spun at 100 rpm for 90 minutes. Simultaneously, a stream of ozone-enriched air was introduced to the bottle; during the reaction time 75.3 mg O₃ was consumed. 500 Milligrams of this material was then transferred to a pressure vessel, and mixed with aqueous ammonium hydroxide (0.107 mL of 28% NH₃ in water) and 0.643 mL of water. The mixture was heated for 90 minutes at 155° C. Upon cooling, the resulting material was dried in vacuo, and 248 mg were suspended in pH 5 buffer (1.500 mL, 14% solids loading). To the resulting slurry Accellerase® 1500 (20.1 μL, 118 mg/mL) and other saccharification enzymes were added, and the mixture was left stirring in an incubator/shaker at 48° C. Samples were taken every 24 h and analyzed by HPLC to generate data on monomeric sugar yields versus time. Results are shown in Tables 7 and 8.

### Comparative Example F

### Control Experiment: Ammonia Treatment for 20 Minutes at 155° C. (No Ozonation)

Switchgrass (500 mg dry, 533 mg wet) was placed into a pressure vessel and mixed with aqueous ammonium hydroxide (0.107 mL of 28% NH₃ in water) and 0.643 mL of water. The mixture was heated for 20 minutes at 155° C. Upon cooling, the resulting material was dried in vacuo, and 256 mg were suspended in pH 5 buffer (1.500 mL, 14% solids loading). To the resulting slurry Accellerase® 1500 (20.7 μL, 118 mg/mL) and other saccharification enzymes were added, and the mixture was left stirring in an incubator/shaker at 48° C. Samples were taken every 24 h and analyzed by HPLC to generate data on monomeric sugar yields versus time. Results are shown in Tables 7 and 8.

### Comparative Example G

### Control Experiment: Ammonia Treatment for 60 Minutes at 155° C. (No Ozonation)

Switchgrass (500 mg dry, 533 mg wet) was placed into a pressure vessel, and mixed with aqueous ammonium hydroxide (0.107 mL of 28% NH₃ in water) and 0.643 mL of water. The mixture was heated for 60 minutes at 155° C. Upon cooling, the resulting material was dried in vacuo, and 249 mg were suspended in pH 5 buffer (1.500 mL, 14% solids loading). To the resulting slurry Accellerase® 1500 (20.1 μL, 118 mg/mL) and other saccharification enzymes were added, and the mixture was left stirring in an incubator/shaker at 48° C. Samples were taken every 24 h and analyzed by HPLC to generate data on monomeric sugar yields versus time. Results are shown in Tables 7 and 8.

### Comparative Example H

### Control Experiment: Ammonia Treatment for 90 Minutes at 155° C. (No Ozonation)

Switchgrass (500 mg dry, 533 mg wet) was placed into a pressure vessel, and mixed with aqueous ammonium hydroxide (0.107 mL of 28% NH₃ in water) and 0.643 mL of water. The mixture was heated for 90 minutes at 155° C. Upon cooling, the resulting material was dried in vacuo, and 253 mg were suspended in pH 5 buffer (1.500 mL, 14% solids loading). To the resulting slurry Accellerase® 1500 (20.5 μL, 118 mg/mL) and other saccharification enzymes were added, and the mixture was left stirring in an incubator/shaker at 48° C. Samples were taken every 24 h and analyzed by HPLC to generate data on monomeric sugar yields versus time. Results are shown in Tables 7 and 8.

TABLE 7
Theoretical yields for glucose during saccharification of biomass
from Examples 10, 11, and 12 and Comparative Examples F,

| Sacch. | Example | Example | Example | Comp. | Comp. | Comp. |
|---|---|---|---|---|---|---|
| Time (h) | 10 | 11 | 12 | Ex. F | Ex. G | Ex. H |
| 24 | 36.9 | 37.4 | 39.5 | 24.1 | 29.1 | 29.1 |
| 48 | 38.5 | 40.1 | 43.7 | 27.2 | 33.6 | 33.8 |
| 72 | 39.9 | 42.2 | 44.6 | 28.8 | 37.7 | 37.5 |
| 96 | 42.5 | 44.8 | 45.7 | 31.2 | 39.4 | 38.0 |
| 120 | 38.5 | 42.8 | 43.9 | 29.7 | 39.1 | 39.1 |
| 144 | 41.7 | 44.2 | 44.9 | 31.6 | 40.6 | 42.1 |

TABLE 8
Theoretical yields for xylose during saccharification of biomass from
Examples 10, 11, and 12 and Comparative Examples F, G, and H.

| Sacch. | Example | Example | Example | Comp. | Comp. | Comp. |
|---|---|---|---|---|---|---|
| Time (h) | 10 | 11 | 12 | Ex. F | Ex. G | Ex. H |
| 24 | 26.5 | 26.5 | 28.4 | 18.3 | 30.2 | 29.9 |
| 48 | 26.8 | 28.2 | 30.2 | 21.1 | 32.9 | 32.7 |
| 72 | 27.4 | 29.2 | 30.6 | 22.3 | 35.8 | 35.5 |
| 96 | 29.2 | 30.6 | 31.0 | 24.2 | 36.6 | 34.9 |
| 120 | 27.2 | 29.9 | 30.3 | 23.6 | 36.7 | 36.2 |
| 144 | 29.4 | 31.0 | 30.7 | 25.1 | 37.8 | 38.7 |

The data show that prolonged ozonolysis and prolonged ammonia pretreatment result in xylose degradation. Hemicellulose, a less recalcitrant component than cellulose, is likely degraded under harsher pretreatment conditions.

Examples 13 through 15 describe pretreatments using 120 minutes of ozonation, and 145° C. ammonia pretreatment for 20, 60, and 90 minutes. Comparative Examples C, D, and E represent their respective controls.

### Example 13

### Effect of Ozonation of Biomass Followed by Ammonia Treatment for 20 Minutes at 145° C.

Switchgrass (7.0 g dry material, adjusted to 60% moisture by addition of water) was placed in a 250 mL bottle and 5 mm Zirconium pellets (30 g) were added. The bottle was placed on a roll mill and spun at 100 rpm for 120 minutes. Simultaneously, a stream of ozone-enriched air was introduced to the bottle; during the reaction time 100.4 mg of O₃ was consumed. 500 Milligrams of the ozone-treated biomass was then transferred to a pressure vessel and mixed with aqueous ammonium hydroxide (0.107 mL of 28% NH₃ in water), and 0.643 mL of water. The mixture was heated for 20 minutes at 145° C. Upon cooling, the resulting material was dried in vacuo, and 251 mg were suspended in pH 5 buffer (1.491 mL, 14% solids loading). To the resulting slurry Accellerase® 1500 (20.3 μL, 118 mg/mL) and other saccharification enzymes were added, and the mixture was left stirring in an incubator/shaker at 48° C. Samples were taken every 24 h and analyzed by HPLC to generate data on monomeric sugar yields versus time. Results are shown in Tables 9 and 10.

### Example 14

### Effect of Ozonation of Biomass Followed by Ammonia Treatment for 60 Minutes at 145° C.

Switchgrass (7.0 g dry material, adjusted to 60% moisture by addition of water) was placed in a 250 mL bottle and 5 mm Zirconium pellets (30 g) were added. The bottle was placed on a roll mill and spun at 100 rpm for 120 minutes. Simultaneously, a stream of ozone-enriched air was introduced to the bottle; during the reaction time 100.0 mg of O₃ was consumed. 500 Milligrams of the ozone-treated biomass was then transferred to a pressure vessel and mixed with aqueous ammonium hydroxide (0.107 mL of 28% NH₃ in water) and 0.643 mL of water. The mixture was heated for 60 minutes at 145° C. Upon cooling, the resulting material was dried in vacuo, and 247 mg were suspended in pH 5 buffer (1.471 mL, 14% solids loading). To the resulting slurry Accellerase® 1500 (20.0 μL, 118 mg/mL) and other saccharification enzymes were added, and the mixture was left stirring in an incubator/shaker at 48° C. Samples were taken every 24 h and analyzed by HPLC to generate data on monomeric sugar yields versus time. Results are shown in Tables 9 and 10.

### Example 15

### Effect of Ozonation of Biomass Followed by Ammonia Treatment for 20 Minutes at 145° C.

Switchgrass (7.0 g dry material, adjusted to 60% moisture by addition of water) was placed in a 250 mL bottle and 5 mm Zirconium pellets (30 g) were added. The bottle was placed on a roll mill and spun at 100 rpm for 120 minutes. Simultaneously, a stream of ozone-enriched air was introduced to the bottle; during the reaction time 75.3 mg O₃ was consumed. 500 Milligrams of the ozone-treated biomass was then transferred to a pressure vessel and mixed with aqueous ammonium hydroxide (0.107 mL of 28% NH₃ in water) and 0.643 mL of water. The mixture was heated for 90 minutes at 145° C. Upon cooling, the resulting material was dried in vacuo, and 247 mg were suspended in pH 5 buffer (1.471 mL, 14% solids loading). To the resulting slurry Accellerase® 1500 (20.0 μL, 118 mg/mL) and other saccharification enzymes were added, and the mixture was left stirring in an incubator/shaker at 48° C. Samples were taken every 24 h and analyzed by HPLC to generate data on monomeric sugar yields versus time. Results are shown in Tables 9 and 10.

TABLE 9
Theoretical yields for glucose during saccharification of biomass
from Examples 13, 14, and 15 and Comparative Examples C, D, and E.

| Sacch. | Example | Example | Example | Comp. | Comp. | Comp. |
|---|---|---|---|---|---|---|
| Time (h) | 13 | 14 | 15 | Ex. C | Ex. D | Ex. E |
| 24 | 39.8 | 39.7 | 39.8 | 20.1 | 20.1 | 24.4 |
| 48 | 44.7 | 45.3 | 44.8 | 23.3 | 23.3 | 29.3 |
| 72 | 43.0 | 43.1 | 44.1 | 24.3 | 24.3 | 31.7 |
| 96 | 45.6 | 45.6 | 45.5 | 26.7 | 28.5 | 33.7 |
| 120 | 47.3 | 47.9 | 47.9 | 26.4 | 29.5 | 35.0 |

TABLE 10
Theoretical yields for xylose during saccharification of biomass from
Examples 13, 14, and 15 and Comparative Examples C, D, and E.

| Sacch. | Example | Example | Example | Comp. | Comp. | Comp. |
|---|---|---|---|---|---|---|
| Time (h) | 13 | 14 | 15 | Ex. C | Ex. D | Ex. E |
| 24 | 28.5 | 30.3 | 31.7 | 13.1 | 13.1 | 20.7 |
| 48 | 30.6 | 32.5 | 33.0 | 16.0 | 16.0 | 24.6 |
| 72 | 30.0 | 31.4 | 32.8 | 17.2 | 17.2 | 26.6 |
| 96 | 33.1 | 33.6 | 33.7 | 19.2 | 21.3 | 28.1 |
| 120 | 35.0 | 34.7 | 34.7 | 19.0 | 21.9 | 29.2 |

Although particular embodiments of the present invention have been described in the foregoing description, it will be understood by those skilled in the art that the invention is capable of numerous modifications, substitutions, and rearrangements without departing from the spirit of essential attributes of the invention. Reference should be made to the appended claims, rather than to the foregoing specification, as indicating the scope of the invention.

What is claimed is:

1. A method for producing readily saccharifiable carbohydrate-enriched biomass, the method comprising:
(a) providing lignocellulosic biomass comprising lignin;
(b) contacting the biomass with an aqueous solution comprising ammonia to form a biomass-aqueous ammonia mixture, wherein the ammonia is present at a concentration at least sufficient to maintain alkaline pH of the biomass-aqueous ammonia mixture but wherein said ammonia is present at less than about 12 weight percent relative to dry weight of biomass, and further wherein the dry weight of biomass is at a high solids concentration of at least about 15 weight percent relative to the weight of the biomass-aqueous ammonia mixture, to produce an ammonia-treated biomass; and
(c) contacting the ammonia-treated biomass with a gas comprising ozone at a temperature of about 0° C. to about 50° C., whereby a readily saccharifiable carbohydrate-enriched biomass is produced.

2. A method for producing readily saccharifiable carbohydrate-enriched biomass, the method comprising:
(a) providing lignocellulosic biomass comprising lignin;
(b) contacting the biomass with a gas comprising ozone at a temperature of about 0° C. to about 50° C.
(c) contacting the ozone-treated biomass with an aqueous solution comprising ammonia to form a mixture comprising ozone-treated biomass and aqueous ammonia, wherein the ammonia is present at a concentration at least sufficient to maintain alkaline pH of the mixture but wherein said ammonia is present at less than about 12 weight percent relative to dry weight of ozone-treated biomass, and further wherein the dry weight of biomass is at a high solids concentration of at least about 15 weight percent relative to the weight of the mixture, whereby a readily saccharifiable carbohydrate-enriched biomass is produced.

3. The method of claim 1 or 2, wherein the gas comprises about 0.1 to about 20 percent by volume ozone.

4. The method of claim 1 or 2, wherein the gas further comprises air, nitrogen, oxygen, argon, or a combination thereof.

5. The method of claim 1, wherein the ratio of ozone to ammonia-treated biomass in step (c) is at least 1:100 on a weight basis.

6. The method of claim 2, wherein the ratio of ozone to lignocellulosic biomass in step (b) is at least 1:100 on a weight basis.

7. The method of claim 1, further comprising applying energy to the lignocellulosic biomass during step (b), to the ammonia-treated biomass during step (c), or to both.

8. The method of claim 2, further comprising applying energy to the lignocellulosic biomass during step (b), to the ozone-treated biomass during step (c), or to both.

9. The method of claim 1 or 2, wherein the lignocellulosic biomass, the ammonia-treated biomass, or both contain at least about 30 percent moisture.

10. The method of claim 1 or 2, wherein ammonia is selected from the group consisting of ammonia gas, ammonium hydroxide, urea, and combinations thereof.

11. The method of claim 1 or 2, wherein the aqueous solution comprising ammonia further comprises at least one additional base selected from the group consisting of sodium hydroxide, sodium carbonate, potassium hydroxide, potassium carbonate, calcium hydroxide, and calcium carbonate.

12. The method of claim 1 or 2, further comprising saccharifying the readily saccharifiable carbohydrate-enriched biomass with an enzyme consortium whereby fermentable sugars are produced.

13. The method of claim 12, further comprising fermenting the sugars to produce a target product.
